SEQUENCE LISTING (84 sequences)

SEQ ID NO: 1 - protein, 538 aa, synthetic construct
MLSQIQRFGGAMFTPVLLFPFAGIVVGLAILLQNPMFVGESLTDPNSLFAQIVHIIEEGGWTVFRNMPLIFAVGLPIGLAKQAQGRACLAVMVSFLTWNYFINAMGMTWGSYFGVDFTQDAVAGSGLTMMAGIKTLDTSIIGAIIISGIVTALHNRLFDKKLPVFLGIFQGTSYVVIIAFLVMIPCAWLTLLGWPKVQMGIESLQAFLRSAGALGVWVYTFLECILIPTGLHHFIYGPFIFGPAAVEGGIQMYWAQHLQEFSLSAEPLKSLFPEGGFALHGNSKIFGAVGISLAMYFTAAPENRVKVAGLLIPATLTAMPVGITEPLEFTFLFISPLLFAVHAVLAASMSTVMYLFSVVGNMGGGLIDQVLPQNWIPMFSNHADMMLTQIAIGLCFTLLYFVVFRTLILQFNMCTPGREDAEVKLYSKAEYKASRGQTTAAEPKKELDQAAGILQALGGVGNISSINNCATRLRIALHDMSQTLDDEVFKKLGAHGVFRSGDAIQVIIGLHVSQLREQLDSLINSHQSAENVAITEAV

SEQ ID NO: 2 - DNA, 1617 bp, synthetic construct
  source 1..1617: mol_type = other DNA
atgctcagtcaaattcaacgctttggcggcgcgatgttcacgccagtgctgctgtttcccttcgccgggattgtggtgggtcttgccatcttgttgcaaaacccgatgtttgtcggggaatcactgaccgatccgaacagtttattcgcgcaaatcgtacacattattgaagagggcggttggacggtattccgtaatatgccgctgatttttgctgtcggtttacccattggccttgctaagcaagcgcaggggcgtgcttgtctggcggtgatggtgagtttcctgacctggaactatttcattaatgcgatgggaatgacctggggaagttacttcggcgtcgatttcactcaggacgcggtggcaggtagcggtctgacaatgatggccgggattaaaaccctcgataccagcattatcggcgcaattatcatttccggcattgtgacggcgctgcataaccgtctgtttgataaaaaactgccggtgtttctcggcattttccaggggacgtcttatgtggtgattatcgccttcctggtgatgatcccctgtgcctggctgacattgctcggctggccaaaagtacaaatggggattgaatctctgcaagcgttcctgcgttcggcgggtgcgcttggggtgtgggtttacaccttcctcgaatgtattctgatcccaaccggtttacaccacttcatctacggaccgtttatctttggtccggcagctgttgaaggcggcattcagatgtactgggcgcagcatctgcaagagttcagtttgagcgccgagccgctgaaatcattgttcccggaaggcggttttgccctgcacggtaactcaaaaatctttggtgccgtgggcatttctttagcgatgtacttcactgccgcaccggaaaatcgggtaaaagtggcgggtttgctgattcccgcaaccttaaccgccatgccggtgggaattaccgaaccgctggaatttaccttcctgttcatttcaccgttgctgtttgcggtacacgctgtgcttgcggcctcaatgtcgaccgtaatgtatctctttagtgtggtgggcaacatgggcggaggtctgattgaccaggttttaccgcaaaactggatcccgatgttcagcaaccacgcggatatgatgctgacccaaatcgccattgggttgtgctttaccctgctgtacttcgtggttttccgcaccctgatactgcagttcaacatgtgtacgccgggacgtgaagatgcggaagtgaaactctactcaaaagccgaatataaagcctcgcgaggccaaaccaccgctgcagagccaaaaaaagagttggatcaggctgccggtatcctgcaagccctgggcggggtcggcaatatctccagcatcaacaattgcgcgacgcgtctacgtattgcactgcatgacatgtcacaaacgctggatgacgaagtctttaaaaagctgggagcgcacggcgtcttccgtagtggcgatgccattcaggtgatcattggtctgcatgtatctcagctgcgtgaacagctcgatagcttaattaattctcatcaatcagcagaaaatgttgccattacggaggcagtataa

SEQ ID NO: 3 - protein, 445 aa, synthetic construct
MSNRKYFGTDGIRGRVGDAPITPDFVLKLGWAAGKVLARHGSRKIIIGKDTRISGYMLESALEAGLAAAGLSALFTGPMPTPAVAYLTRTFRAEAGIVISASHNPFYDNGIKFFSIDGTKLPDAVEETIEAEMEKEISCVDSAELGKASRIVDAAGRYIEFCKATFPNELSLSELKIVVDCANGATYHIAPNVLRELGANVIAIGCEPNGVNINAEVGATDVRALQARVLAEKADLGIAFDGDGDRVIMVDHEGNKVDGDQIMYIIAREGLRQGQLRGGAVGTLMSNMGLELALKQLGIPFARAKVGDRYVLEKMQEKGWRIGAENSGHVILLDKTTTGDGIVAGLQVLAAMARNHMSLHDLCSGMKMFPQILVNVRYTAGSGDPLEHESVKAVTAEVEAALGNRGRVLLRKSGTEPLIRVMVEGEDEAQVTEFAHRIADAVKAV

SEQ ID NO: 4 - DNA, 1338 bp, synthetic construct
  source 1..1338: mol_type = other DNA
atgagtaatcgtaaatatttcggtaccgatgggattcgtggtcgtgtaggggatgcgccgatcacacctgattttgtgcttaagctgggttgggccgcgggtaaagtgctggcgcgccacggctcccgtaagatcattattggtaaagacacgcgtatttctggctatatgctggagtcagcactggaagcgggtctggcggcagcgggcctttccgcactcttcactggcccgatgccaacaccggccgtggcttatctgacgcgtaccttccgcgcagaggccggaattgtgatatctgcatcgcataacccgttctacgataatggcattaaattcttctctatcgacggcaccaaactgccggatgcggtagaagagaccatcgaagcggaaatggaaaaggagatcagctgcgttgattcggcagaactgggtaaagccagccgtatcgttgatgccgcgggtcgctatatcgagttttgcaaagccacgttcccgaacgaacttagcctcagtgaactgaagattgtggtggattgtgcaaacggtgcgacttatcacatcgcgccgaacgtgctgcgcgaactgggggcgaacgttatcgctatcggttgtgagccaaacggtgtaaacatcaatgccgaagtgggggctaccgacgttcgcgcgctccaggctcgtgtgctggctgaaaaagcggatctcggtattgccttcgacggcgatggcgatcgcgtgattatggttgaccatgaaggcaataaagtcgatggcgatcagatcatgtatatcatcgcgcgtgaaggtcttcgtcagggccagctgcgtggtggcgctgtgggtacattgatgagcaacatggggcttgaactggcgctgaaacagttaggaattccatttgcgcgcgcgaaagtgggtgaccgctacgtactggaaaaaatgcaggagaaaggctggcgtatcggtgcagagaattccggtcatgtgatcctgctggataaaactactaccggtgacggcatcgttgctggcttgcaggtgctggcggctatggcacgtaaccatatgagcctgcacgacctttgcagcggcatgaaaatgttcccgcagattctggttaacgtacgttacaccgcaggtagcggcgatccacttgagcatgagtcagttaaagccgtgaccgcagaggttgaagctgcgctgggcaaccgtggacgcgtgttgctgcgtaaatccggcaccgaaccgttaattcgcgtgatggtggaaggcgaagacgaagcgcaggtgactgaatttgcacaccgcatcgccgatgcagtaaaagccgtttaa

SEQ ID NO: 5 - protein, 1517 aa, synthetic construct
MTRKPRRHALSVPVRSGSEVGFPQSLGEVHDMLYDKSLERDNCGFGLIAHIEGEPSHKVVRTAIHALARMQHRGAILADGKTGDGCGLLLQKPDRFFRIVAQERGWRLAKNYAVGMLFLNKDPELAAAARRIVEEELQRETLSIVGWRDVPTNEGVLGEIALSSLPRIEQIFVNAPAGWRPRDMERRLFIARRRIEKRLEADKDFYVCSLSNLVNIYKGLCMSADLPRFYLDLADLRLESAICLFHQRFSTNTVPRWPLAQPFRYLAHNGEINTITGNRQWARARTYKFQTPLIPDLHDAAPFVNETGSDSSSMDNMLELLLAGGMDIIRAMRLLVPPAWQNNPDMDPELRAFFDFNSMHMEPWDGPAGIVMSDGRFAACNLDRNGLRPARYVITKDKLITCASEVGIWDYQPDEVVEKGRVGPGELMVIDTRSGRILHSAETDDDLKSRHPYKEWMEKNVRRLVPFEDLPDEEVGSRELDDDTLASYQKQFNYSAEELDSVIRVLGENGQEAVGSMGDDTPFAVLSSQPRIIYDYFRQQFAQVTNPPIDPLREAHVMSLATSIGREMNVFCEAEGQAHRLSFKSPILLYSDFKQLTTMKEEHYRADTLDITFDVTKTTLEATVKELCDKAEKMVRSGTVLLVLSDRNIAKDRLPVPAPMAVGAIQTRLVDQSLRCDANIIVETASARDPHHFAVLLGFGATAIYPYLAYETLGRLVDTHAIAKDYRTVMLNYRNGINKGLYKIMSKMGISTIASYRCSKLFEAVGLHDDVVGLCFQGAVSRIGGASFEDFQQDLLNLSKRAWLARKPISQGGLLKYVHGGEYHAYNPDVVRTLQQAVQSGEYSDYQEYAKLVNERPATTLRDLLAITPGENAVNIADVEPASELFKRFDTAAMSIGALSPEAHEALAEAMNSIGGNSNSGEGGEDPARYGTNKVSRIKQVASGRFGVTPAYLVNADVIQIKVAQGAKPGEGGQLPGDKVTPYIAKLRYSVPGVTLISPPPHHDIYSIEDLAQLIFDLKQVNPKAMISVKLVSEPGVGTIATGVAKAYADLITIAGYDGGTGASPLSSVKYAGCPWELGLVETQQALVANGLRHKIRLQVDGGLKTGVDIIKAAILGAESFGFGTGPMVALGCKYLRICHLNNCATGVATQDDKLRKNHYHGLPFKVTNYFEFIARETRELMAQLGVTRLVDLIGRTDLLKELDGFTAKQQKLALSKLLETAEPHPGKALYCTENNPPFDNGLLNAQLLQQAKPFVDERQSKTFWFDIRNTDRSVGASLSGYIAQTHGDQGLAADPIKAYFNGTAGQSFGVWNAGGVELYLTGDANDYVGKGMAGGLIAIRPPVGSAFRSHEASIIGNTCLYGATGGRLYAAGRAGERFGVRNSGAITVVEGIGDNGCEYMTGGIVCILGKTGVNFGAGMTGGFAYVLDESGDFRKRVNPELVEVLSVDALAIHEEHLRGLITEHVQHTGSQRGEEILANWSTFATKFALVKPKSSDVKALLGHRSRSAAELRVQAQ

SEQ ID NO: 6 - DNA, 4554 bp, synthetic construct
  source 1..4554: mol_type = other DNA
atgacacgcaaaccccgtcgccacgctctttctgtgcccgtgcgcagcggttcggaagtggggttcccgcagagcctgggggaggttcacgatatgttgtacgataaatcccttgagagggataactgtggtttcggcctgatcgcccacatagaaggcgaacctagccacaaggtagtgcgtactgcaatacacgcactggcccgcatgcagcaccgtggcgcgattctcgccgatggtaaaaccggtgacggttgcggcttgctgttacaaaaaccggatcgcttttttcgcatcgttgcgcaggagcgcggctggcgtttagcgaaaaactacgccgtcgggatgctattcctgaataaagatcctgaactcgccgctgccgcacgccgcatcgttgaagaagagctgcaacgcgaaaccttgtcgattgtgggctggcgtgatgtccccactaacgaaggcgtgctgggtgaaatcgccctctcctctctgccacgcattgagcaaatttttgtgaacgccccggcaggctggcgtccacgcgatatggagcgccgtctgtttatcgcccgccgccgcattgaaaagcgtctcgaagccgacaaagacttctacgtctgtagcctgtcgaatctggtgaacatctataaaggtctgtgtatgtcggcggatctgccgcgcttttatctggatcttgcggacctgcgtctggaatcggccatttgcctgttccaccagcgcttctccactaacaccgtaccgcgctggccgctggcgcaaccgttccgctatctggcgcataacggtgaaatcaacaccatcaccggtaaccgccaatgggcgcgtgcgcgtacctataaattccagacgccgcttatccctgatctgcacgacgccgcaccgttcgtcaacgaaaccggctctgactccagttccatggataacatgctggaactgctgctggcaggcgggatggatatcatccgcgccatgcgtctattagtaccacccgcctggcagaacaacccggatatggacccggaactgcgtgccttctttgactttaactccatgcatatggagccgtgggatggcccggcgggcatcgtgatgtccgacggtcgttttgccgcctgtaacctcgaccgtaacggtctgcgtccggcgcgctacgtcatcaccaaagataagctcatcacctgcgcctctgaagtcggtatctgggattaccagcctgacgaagtggtcgaaaaaggccgcgtcgggccaggcgaactgatggttatcgacacccgcagtgggcgtattctgcactcggcagaaaccgatgacgatctgaaaagccgccatccatataaagagtggatggagaaaaacgtccgccgactggtaccgtttgaagatctgcccgatgaagaagtgggtagccgcgaactggacgacgacacgcttgccagctaccagaaacagtttaactacagcgcggaagagctggactccgtaattcgcgtactgggcgaaaacggtcaggaagcggtcggttcgatgggcgatgataccccattcgccgtgctctccagtcagccgcgcattatttacgactacttccgccagcagtttgcccaggtgactaacccgccaatcgacccgctgcgtgaagcgcatgttatgtcgctcgccaccagtatcggtcgtgaaatgaacgtcttttgcgaagcagagggccaggcgcaccgtttaagctttaaatcgccgattctgctctactccgatttcaaacagctcacgacgatgaaagaggagcactaccgcgcagatacgctggatatcacctttgacgtcactaaaaccacgctcgaagcgacagtcaaagagctgtgcgacaaagccgaaaaaatggtacgtagcggcaccgtgctgctggtgctctccgaccggaatatcgctaaagatcgcctgccggttccagccccgatggcggttggcgcgatccagacccgtctggtcgatcaaagcctgcgttgcgatgccaacatcatcgtcgaaaccgccagcgcccgcgatccgcaccacttcgccgtgttgctgggcttcggcgcgacggctatttatccataccttgcctatgaaacgctgggccgcctggtagacacccatgcgattgccaaagattatcgtaccgtgatgctcaactaccgtaacggcatcaacaaaggcttgtacaaaatcatgtccaaaatgggcatctccaccatcgcctcttaccgctgctcgaaactgtttgaagcggtcggtctacacgatgatgtagtgggcctgtgcttccagggggcggtcagccgcattggtggggcaagctttgaagacttccagcaggatctgctgaatctgtcgaaacgtgcctggctggcgcgtaagcccatcagccagggcggcctgctgaaatacgtccacggcggcgaataccacgcctacaacccggacgtggtgcgcacgctgcaacaagcggtacaaagcggcgagtacagcgactatcaggaatacgcgaagctggttaatgagcgtccggcaaccacgctgcgcgatctgctggcaattacgccgggtgaaaacgcggtcaacattgctgatgttgaaccggcaagcgaactgtttaaacgctttgataccgccgcgatgtctatcggcgcgttaagcccggaagcccacgaggcgctggcggaagcgatgaacagcatcggcggtaattcgaactccggtgaaggcggcgaagacccggcgcgctacggcaccaacaaagtgtcgcgcatcaagcaggtggcttccggtcgctttggggttactccggcgtatctggtcaatgccgacgtcattcagattaaagtcgcccagggcgcgaagccaggcgaaggcggtcagttgccgggtgataaagtcacgccttacatcgccaaactgcgctattcggtgcccggagtgacgctgatctccccgccgccgcaccacgatatctactctatcgaggacttagcgcagctcattttcgacctcaagcaggttaacccgaaagcgatgatctccgtgaagctggtttccgaaccgggagtaggcaccatcgcgactggcgtggcaaaagcttatgcggacttgatcaccatcgcaggctatgacggcggcaccggcgcaagtccgctttcatcggtgaaatacgcaggctgtccgtgggagctggggcttgttgaaacccagcaggcgctggttgctaacggcttgcgtcacaagatccgtttgcaggtcgatggcggcctgaaaacgggtgtcgatatcatcaaggcggcgattctcggcgcagaaagcttcggcttcggcactggcccgatggtggcgctcggctgtaaatatctacgtatttgccatctgaacaactgcgcaacgggtgtagcaactcaggatgacaaactgcgtaagaaccactatcacggcctgccattcaaggtgacgaattactttgagtttatcgcccgtgaaacccgcgagctgatggcacagcttggcgtaacacgtctggtggatctgattggtcgcaccgacctgctgaaagagctggacggtttcaccgccaaacagcagaagctggcgctgtcgaagctgctggagactgccgaaccgcatccaggtaaggcactctactgcaccgaaaacaacccgccgtttgataacggcctgctgaacgcgcagttgctgcaacaggcgaaaccgtttgtcgatgagcgccagagcaaaaccttctggttcgatattcgcaacaccgaccgttctgtcggcgcgtcgctttcaggctatatcgcccagacgcacggcgatcaggggctggcagccgatcctatcaaagcgtacttcaacggcaccgcaggccagagcttcggcgtgtggaacgcgggcggcgtggaactgtacctgaccggtgatgccaacgactatgtcggtaaaggcatggcgggcggcttaatcgccattcgtcctccggttggttccgccttccgcagccatgaagcaagcattatcggcaacacctgcctgtatggcgcgaccggtggtcgtctgtatgccgcaggccgcgcgggtgaacgtttcggcgtgcgtaactccggtgctatcaccgtggtagaaggcattggcgacaacggttgtgaatatatgacgggtggtatcgtctgcattctgggtaaaaccggcgttaacttcggtgcgggcatgaccggcggtttcgcttacgttctcgatgaaagcggcgatttccgcaaacgcgttaacccggaactggtcgaggtcttaagcgttgacgctctggcgatccatgaagagcatctgcgtggtcttatcaccgagcatgtgcagcataccggctctcagcgcggtgaagagattctggcgaactggtcaaccttcgccactaaatttgcgctggttaaaccgaagtccagtgatgtaaaagcactgctgggtcaccgtagtcgtagcgcagctgagttgcgcgtgcaggcgcagtaa

SEQ ID NO: 7 - protein, 472 aa, synthetic construct
MSQNVYQFIDLQRVDPPKKPLKIRKIEFVEIYEPFSEGQAKAQADRCLSCGNPYCEWKCPVHNYIPNWLKLANEGRIFEAAELSHQTNTLPEVCGRVCPQDRLCEGSCTLNDEFGAVTIGNIERYINDKAFEMGWRPDMSGVKQTGKKVAIIGAGPAGLACADVLTRNGVKAVVFDRHPEIGGLLTFGISAFKLEKEVMTRRREIFTGMGIEFKLNTEVGRDVQLDDLLSDYDAVFLGVGTYQSMHGGLENEDADGVYAALPFLIANTKQLMGFGETRDEPFVSMKGKRVVVLGGGDTAMDCVRTSVRQGAKHVTCAYRRDEENMPGSRREVKNAREEGVEFKFNVQPLGIEVNGNGKVSGVKMVRTEMGEPDAKGRRRAEIVAGSEHIVPADAVIMAFGFRPHNMEWLAKHSVELDSQGRIIAPEGSDNAFQTSNPKIFAGGDIVRGSDLVVTAIAEGRKAADGIMNWLEV

SEQ ID NO: 8 - DNA, 1419 bp, synthetic construct
  source 1..1419: mol_type = other DNA
atgagtcagaatgtttatcaatttatcgacctgcagcgcgttgatccgccaaagaaaccgctgaagatccgcaaaattgagtttgttgaaatttacgagccgttttccgaaggccaggccaaagcgcaggctgaccgctgcctgtcgtgcggcaacccatactgcgagtggaaatgcccggtacacaactacatcccgaactggctgaagctagccaacgaggggcgtatttttgaagcggcggaactgtcgcaccagaccaacaccctgccggaagtttgcggacgagtctgcccgcaagaccgtctgtgcgaaggttcctgcactctgaacgatgagtttggcgcggtgaccatcggcaacattgagcgctatatcaacgataaagcgttcgagatgggctggcgtccggatatgtctggtgtgaaacagaccggtaaaaaagtggcgattatcggcgcaggcccggcaggtctggcgtgtgcggatgtcctgacgcgtaacggcgtaaaagccgttgtcttcgaccgtcatccagaaattggcgggctgctgaccttcggtatttcggccttcaagctggaaaaagaggtaatgacgcgtcgccgtgaaatcttcaccggcatgggtattgaattcaaactcaataccgaagtgggccgcgacgtgcagctggacgatctgctgagtgattacgatgccgtcttccttggcgtcgggacttatcagtcaatgcacggcgggctggaaaacgaagacgccgatggcgtgtacgcagcgctgccgttccttatcgccaacaccaaacagttaatgggctttggtgaaacccgcgacgaaccgttcgtcagcatgaaaggcaaacgcgtggtggtccttggcggtggcgacactgcgatggactgcgtgcgtacgtccgtgcgccagggagcgaagcacgttacctgtgcctatcgtcgtgatgaagagaacatgccgggttcccgccgcgaagtgaaaaacgcgcgggaagaaggcgtagagttcaaattcaacgtccagccactggggattgaagtgaacggtaacggcaaagtcagcggcgtaaaaatggtacgtaccgaaatgggcgaaccggatgccaaaggccgtcgccgcgcggagatcgttgcaggttccgaacatatcgttccggcagatgcggtgatcatggcgtttggtttccgtccacacaacatggaatggctggcaaaacacagcgtcgagctggattcacaaggccgcatcatcgccccggaaggcagcgacaacgccttccagaccagcaacccgaaaatctttgctggcggcgatatcgtccgtggttccgatctggtggtaaccgctattgccgaaggtcgtaaggcggcagacggtattatgaactggctggaagtttaa

SEQ ID NO: 9 - protein, 322 aa, synthetic construct
MTMPTNQCPWRMQVHHITQETPDVWTISLICHDYYPYRAGQYALVSVRNSAETLRAYTISSTPGVSEYITLTVRRIDDGVGSQWLTRDVKRGDYLWLSDAMGEFTCDDKAEDKFLLLAAGCGVTPIMSMRRWLAKNRPQADVRVIYNVRTPQDVIFADEWRNYPVTLVAENNVIEGFIAGRLTRELLAGVPDLASRTVMTCGPAPYMDWVEQEVKALGVTRFFKEKFFTPVAEAATSGLKFTKLQPAREFYAPVGTTLLEALESNNVPVVAACRAGVCGCCKTKVVSGEYTVSSTMTLTDAEIAEGYVLACSCHPQGDLVLA

SEQ ID NO: 10 - DNA, 969 bp, synthetic construct
  source 1..969: mol_type = other DNA
atgacgatgccaacgaatcaatgcccgtggcggatgcaggttcatcacattacgcaagaaacgccggatgtgtggacgatttccctgatttgccacgattactacccatatcgcgccgggcaatatgcactggtcagcgtgcgtaactcagcggaaacgctgcgtgcttacaccatttcctccacgccaggagtgagtgaatacatcaccctgaccgtgcggcggattgatgacggtgtcggctcccagtggctgacgcgcgatgtaaaacgcggtgattatctctggctttcggacgcgatgggggaatttacctgcgacgataaagcagaagataaattcctgttgctggcggcaggctgcggcgtcacgccgattatgtcgatgcgtcgctggcttgcgaagaaccgtccacaggccgatgtgcgggtgatctacaacgtgcgtacgccgcaggatgttattttcgccgatgagtggcgtaactatccggtaacgctggtggcagaaaataacgttatcgaaggctttatcgctggtcgtctcactcgcgaactgctggcaggtgtacctgacttagcttcacgtaccgtgatgacctgcggcccggctccgtatatggattgggtagagcaggaagtgaaagcgctcggcgtgacgcgtttctttaaagagaaattcttcaccccagtagcagaagcggcgaccagcggtctgaaattcaccaaactgcaaccggcacgagaattttacgccccggttggcaccacgctactggaggcgctggaaagcaataacgttccggttgtcgctgcctgccgtgcgggtgtttgcggctgctgtaagacaaaagtggtttccggtgaatatacggtgagcagcacaatgacgctgaccgacgccgaaatcgctgaaggttacgtactggcctgctcctgccatccgcagggggatttggttctcgcataa

SEQ ID NO: 11 - protein, 211 aa, synthetic construct
MFKRLMMVALLVIAPLSAATAADQTNPYKLMDEAAQKTFDRLKNEQPQIRANPDYLRTIVDQELLPYVQVKYAGALVLGQYYKSATPVQREAYFAAFREYLKQAYGQALAMYHGQTYQIAPEQPLGDKTIVPIRVTIIDSNGRPPVRLDFQWRKNSQTGNWQAYDMIAEGVSMITTKQNEWGTLLRTKGIDGLTAQLKSISQQKITLEEKK

SEQ ID NO: 12 - DNA, 636 bp, synthetic construct
  source 1..636: mol_type = other DNA
atgtttaaacgtttaatgatggtcgctttgctggtgattgcacctctgagtgcggcaaccgcggcagaccagaccaatccgtataagctgatggacgaggcggcgcagaaaacgttcgatcgcctgaagaatgagcaaccgcaaattcgggccaacccggattatctgcgtaccattgttgatcaggaactgctgccatacgtacaggtgaaatacgccggtgcgctggtgctgggccagtattacaagagtgcgacccctgttcaacgtgaagcctactttgccgctttccgtgagtacctgaagcaggcttacggtcaggcgctggcgatgtatcacggtcaaacctatcagattgcgccagaacagccgctgggcgataaaaccattgtgcctattcgcgttaccattattgactcgaatggccgtccgccggtgcgtctggacttccagtggcgtaaaaactcccagacgggcaattggcaggcttacgacatgattgctgaaggcgtcagtatgatcaccaccaaacaaaacgagtggggaacgctgctgcgtaccaaaggtatcgacggcctgactgcgcaactgaaatcgatttctcaacagaaaatcactctggaagagaaaaaataa

SEQ ID NO: 13 - DNA, 20 bp, synthetic construct
  source 1..20: mol_type = other DNA
ggccttgctaagcaagcgca

SEQ ID NO: 14 - DNA, 20 bp, synthetic construct
  source 1..20: mol_type = other DNA
attgggtcgtcatgttctgc

SEQ ID NO: 15 - DNA, 20 bp, synthetic construct
  source 1..20: mol_type = other DNA
ctttcgcgcgcgcaaatgga

SEQ ID NO: 16 - DNA, 20 bp, synthetic construct
  source 1..20: mol_type = other DNA
tttaatctttcccactgaag

SEQ ID NO: 17 - DNA, 20 bp, synthetic construct
  source 1..20: mol_type = other DNA
gatgcggcgcggtacgtacg

SEQ ID NO: 18 - DNA, 20 bp, synthetic construct
  source 1..20: mol_type = other DNA
ttatcgcgcccatggccgaa

SEQ ID NO: 19 - DNA, 20 bp, synthetic construct
  source 1..20: mol_type = other DNA
ccgtgaaatcttcaccggca

SEQ ID NO: 20 - DNA, 20 bp, synthetic construct
  source 1..20: mol_type = other DNA
cttcgccactaaatttgcgc

SEQ ID NO: 21 - DNA, 19 bp, synthetic construct
  source 1..19: mol_type = other DNA
ttatgtcgatgcgtcgctg

SEQ ID NO: 22 - DNA, 20 bp, synthetic construct
  source 1..20: mol_type = other DNA
gactgcacctggtttcctga

SEQ ID NO: 23 - DNA, 20 bp, synthetic construct
  source 1..20: mol_type = other DNA
tggaagtccagacgcaccgg

SEQ ID NO: 24 - DNA, 20 bp, synthetic construct
  source 1..20: mol_type = other DNA
tcctcacgcatttcccaaag

SEQ ID NO: 25 - DNA, 21 bp, synthetic construct
  source 1..21: mol_type = other DNA
aatggccggaggcgcaggcat

SEQ ID NO: 26 - DNA, 44 bp, synthetic construct
  source 1..44: mol_type = other DNA
aaccactgagaatttggtcattccacatcctcttttctcaattc

SEQ ID NO: 27 - DNA, 24 bp, synthetic construct
  source 1..24: mol_type = other DNA
tgaccaaattctcagtggttgtcg

SEQ ID NO: 28 - DNA, 23 bp, synthetic construct
  source 1..23: mol_type = other DNA
ggctttcaataccgattggcatg

SEQ ID NO: 29 - DNA, 23 bp, synthetic construct
  source 1..23: mol_type = other DNA
cgcggatgttagccaaaatgaat

SEQ ID NO: 30 - DNA, 22 bp, synthetic construct
  source 1..22: mol_type = other DNA
cttcaattgatgtccaccagcc

SEQ ID NO: 31 - DNA, 20 bp, synthetic construct
  source 1..20: mol_type = other DNA
cacctggtattcgctggcat

SEQ ID NO: 32 - DNA, 46 bp, synthetic construct
  source 1..46: mol_type = other DNA
cgttgaatttgactgagcataagcattgcggagcggttaagaattc

SEQ ID NO: 33 - DNA, 44 bp, synthetic construct
  source 1..44: mol_type = other DNA
ccattacggaggcagtataatgaccaaattctcagtggttgtcg

SEQ ID NO: 34 - DNA, 23 bp, synthetic construct
  source 1..23: mol_type = other DNA
ggctttcaataccgattggcatg

SEQ ID NO: 35 - DNA, 22 bp, synthetic construct
  source 1..22: mol_type = other DNA
ggatatcgctggtcggcttagc

SEQ ID NO: 36 - DNA, 45 bp, synthetic construct
  source 1..45: mol_type = other DNA
tgcaaaggaaaacaaacgctagcgttagataactggctaaaaagg

SEQ ID NO: 37 - DNA, 23 bp, synthetic construct
  source 1..23: mol_type = other DNA
agcgtttgttttcctttgcagac

SEQ ID NO: 38 - DNA, 23 bp, synthetic construct
  source 1..23: mol_type = other DNA
acatccaaaccagaagtcatccg

SEQ ID NO: 39 - DNA, 23 bp, synthetic construct
  source 1..23: mol_type = other DNA
atccatatcagaccgaaacactg

SEQ ID NO: 40 - DNA, 20 bp, synthetic construct
  source 1..20: mol_type = other DNA
ggcggcggaagttagcgttg

SEQ ID NO: 41 - DNA, 21 bp, synthetic construct
  source 1..21: mol_type = other DNA
ccggaactggagttgacacaa

SEQ ID NO: 42 - DNA, 39 bp, synthetic construct
  source 1..39: mol_type = other DNA
atgcagtaaaagccgtttaaaggcgctgatatgggagcc

SEQ ID NO: 43 - DNA, 43 bp, synthetic construct
  source 1..43: mol_type = other DNA
aaatatttacgattactcatagcgtttgttttcctttgcagac

SEQ ID NO: 44 - DNA, 23 bp, synthetic construct
  source 1..23: mol_type = other DNA
acatccaaaccagaagtcatccg

SEQ ID NO: 45 - DNA, 20 bp, synthetic construct
  source 1..20: mol_type = other DNA
gtgcgcgggatgattccagt

SEQ ID NO: 46 - DNA, 40 bp, synthetic construct
  source 1..40: mol_type = other DNA
gttaactgagaacaaactaatcgtctgaaagctggcagcc

SEQ ID NO: 47 - DNA, 29 bp, synthetic construct
  source 1..29: mol_type = other DNA
ttagtttgttctcagttaacaattcatat

SEQ ID NO: 48 - DNA, 22 bp, synthetic construct
  source 1..22: mol_type = other DNA
attttttcaggccggatatggc

SEQ ID NO: 49 - DNA, 19 bp, synthetic construct
  source 1..19: mol_type = other DNA
ccgcgcgtctgtttgaggc

SEQ ID NO: 50 - DNA, 22 bp, synthetic construct
  source 1..22: mol_type = other DNA
attttttcaggccggatatggc

SEQ ID NO: 51 - DNA, 22 bp, synthetic construct
  source 1..22: mol_type = other DNA
ggtaaaaacgtcggcgacgatc

SEQ ID NO: 52 - DNA, 52 bp, synthetic construct
  source 1..52: mol_type = other DNA
agcgtgtgaaaggcgagtaatactttccatctttcctcactccttaagttag

SEQ ID NO: 53 - DNA, 49 bp, synthetic construct
  source 1..49: mol_type = other DNA
tgaacacgaaatttatccatttagtttgttctcagttaacaattcatat

SEQ ID NO: 54 - DNA, 22 bp, synthetic construct
  source 1..22: mol_type = other DNA
attttttcaggccggatatggc

SEQ ID NO: 55 - DNA, 20 bp, synthetic construct
  source 1..20: mol_type = other DNA
tttcggcgtgcgtaactccg

SEQ ID NO: 56 - DNA, 41 bp, synthetic construct
  source 1..41: mol_type = other DNA
aggcggcgctgggacagagctgttgctaccccttactgcgc

SEQ ID NO: 57 - DNA, 20 bp, synthetic construct
  source 1..20: mol_type = other DNA
gctctgtcccagcgccgcct

SEQ ID NO: 58 - DNA, 21 bp, synthetic construct
  source 1..21: mol_type = other DNA
aattcgccagacactgtctgg

SEQ ID NO: 59 - DNA, 20 bp, synthetic construct
  source 1..20: mol_type = other DNA
agactgccgaaccgcatcca

SEQ ID NO: 60 - DNA, 21 bp, synthetic construct
  source 1..21: mol_type = other DNA
ttccacctctagaaggtcgtg

SEQ ID NO: 61 - DNA, 20 bp, synthetic construct
  source 1..20: mol_type = other DNA
tgataacggcctgctgaacg

SEQ ID NO: 62 - DNA, 39 bp, synthetic construct
  source 1..39: mol_type = other DNA
tgataaacattctgactcatgcagacgataccacccgtc

SEQ ID NO: 63 - DNA, 40 bp, synthetic construct
  source 1..40: mol_type = other DNA
tgaactggctggaagtttaagctctgtcccagcgccgcct

SEQ ID NO: 64 - DNA, 21 bp, synthetic construct
  source 1..21: mol_type = other DNA
aattcgccagacactgtctgg

SEQ ID NO: 65 - DNA, 22 bp, synthetic construct
  source 1..22: mol_type = other DNA
ggtgagccgtgaaaaactgcgt

SEQ ID NO: 66 - DNA, 40 bp, synthetic construct
  source 1..40: mol_type = other DNA
tatcatcgggcataaggcgacgacaaactccttacgcgct

SEQ ID NO: 67 - DNA, 25 bp, synthetic construct
  source 1..25: mol_type = other DNA
tcgccttatgcccgatgatattcct

SEQ ID NO: 68 - DNA, 22 bp, synthetic construct
  source 1..22: mol_type = other DNA
gcaatcgccagtacttgtggga

SEQ ID NO: 69 - DNA, 20 bp, synthetic construct
  source 1..20: mol_type = other DNA
tggatgcaggcgaaaccggt

SEQ ID NO: 70 - DNA, 20 bp, synthetic construct
  source 1..20: mol_type = other DNA
gcatgataccagtgcatggt

SEQ ID NO: 71 - DNA, 21 bp, synthetic construct
  source 1..21: mol_type = other DNA
gcgaaatgctgcctgcgcatg

SEQ ID NO: 72 - DNA, 40 bp, synthetic construct
  source 1..40: mol_type = other DNA
ttgattcgttggcatcgtcatagacgcggcagaccttcga

SEQ ID NO: 73 - DNA, 45 bp, synthetic construct
  source 1..45: mol_type = other DNA
gggatttggttctcgcataatcgccttatgcccgatgatattcct

SEQ ID NO: 74 - DNA, 22 bp, synthetic construct
  source 1..22: mol_type = other DNA
gcaatcgccagtacttgtggga

SEQ ID NO: 75 - DNA, 20 bp, synthetic construct
  source 1..20: mol_type = other DNA
ctgaaagtggctgccatcgc

SEQ ID NO: 76 - DNA, 41 bp, synthetic construct
  source 1..41: mol_type = other DNA
aatttcaggagaaccgacgctgagcgagtcactgagctgga

SEQ ID NO: 77 - DNA, 22 bp, synthetic construct
  source 1..22: mol_type = other DNA
gcgtcggttctcctgaaattat

SEQ ID NO: 78 - DNA, 21 bp, synthetic construct
  source 1..21: mol_type = other DNA
tattagcagcactgctggcgg

SEQ ID NO: 79 - DNA, 29 bp, synthetic construct
  source 1..29: mol_type = other DNA
ttagtttgttctcagttaacaattcatat

SEQ ID NO: 80 - DNA, 22 bp, synthetic construct
  source 1..22: mol_type = other DNA
atttcgttgtttaacggctacg

SEQ ID NO: 81 - DNA, 29 bp, synthetic construct
  source 1..29: mol_type = other DNA
ttagtttgttctcagttaacaattcatat

SEQ ID NO: 82 - DNA, 42 bp, synthetic construct
  source 1..42: mol_type = other DNA
ctctggaagagaaaaaataaattttttcaggccggatatggc

SEQ ID NO: 83 - DNA, 42 bp, synthetic construct
  source 1..42: mol_type = other DNA
atcattaaacgtttaaacatgcgtcggttctcctgaaattat

SEQ ID NO: 84 - DNA, 21 bp, synthetic construct
  source 1..21: mol_type = other DNA
tattagcagcactgctggcgg

(19) United States
(12) Patent Application Publication
(10) Pub. No.: US 2026/0286412 A1
(43) Pub. Date: Sep. 24, 2026

(54) High-yield fructosylated chondroitin-producing Escherichia coli and its application thereof

(71) Applicants: Yantai Dongcheng Biochemicals Co., Ltd, Yantai (CN); Jiangnan University, Wuxi (CN)

(72) Inventors: De YANG, Yantai (CN); Xiaohang LIU, Yantai (CN); Yimin XIN, Yantai (CN); Nan WANG, Yantai (CN); Jing WU, Wuxi (CN)

(21) Appl. No.: 19/632,920

(22) Filed: Mar. 30, 2026

Related U.S. Application Data
(63) Continuation of application No. PCT/CN2025/101665, filed on Jun. 18, 2025.

(30) Foreign Application Priority Data
Mar. 18, 2025 (CN) 2025103149980

(51) Int. Cl. C12P 19/04; C07K 14/245; C12N 9/02; C12N 9/06; C12N 9/12; C12N 9/90

(52) U.S. Cl. CPC C12P 19/04; C07K 14/245; C12N 9/0004; C12N 9/0016; C12N 9/1205; C12N 9/90; C12Y 104/01013; C12Y 504/0201

(57) ABSTRACT

The present disclosure discloses a high-yield fructosylated chondroitin-producing Escherichia coli and its application thereof, belonging to the technical field of microbiology. On the basis of E. coli K4, the present disclosure constructs a strain with enhanced fructosylated chondroitin synthesis capacity by mutating genes encoding key enzymes in a fructosylated chondroitin synthesis pathway. The yield of fructosylated chondroitin produced by this strain reaches 10 g/L in a 5 L fermenter and 15-20 g/L in a 100 L fermenter. The obtained final product has a uniform structure and stable quality, which effectively reduces the production costs and the risk of environmental pollution, thus being suitable for large-scale industrial production applications.

The instant application contains a Sequence Listing in XML format as a file named “3050-YGHY-2025-37-SEQ.xml”, created on Mar. 18, 2026, of 89,343 bytes in size, and which is hereby incorporated by reference in its entirety.

### TECHNICAL FIELD

The present disclosure relates to a high-yield fructosylated chondroitin-producing Escherichia coli and its application thereof, belonging to the technical field of microbiology.

### BACKGROUND

Chondroitin sulfate is a widely distributed sulfated glycosaminoglycan found in animal tissues, where a chondroitin backbone is formed by the linear alternating linkage of glucuronic acid (GlcUA) and N-acetylgalactosamine (GalNAc) via 1,3-bonds and 1,4-bonds, respectively. At present, chondroitin sulfate is mainly extracted directly from animal tissues. This method suffers from drawbacks such as long production cycles and scattered distribution of raw materials, cumbersome and time-consuming process steps, as well as unstable product quality and yield caused by raw material variations, so that microbial fermentation methods with mild conditions and abundant raw materials present a more attractive alternative for chondroitin sulfate production. Analogs of chondroitin are attached to cell walls of certain prokaryotic microorganisms in the form of capsular polysaccharides. For instance, the capsular polysaccharide of Escherichia coli K4 is a chondroitin backbone modified with fructosyl groups. As an emerging strategy for chondroitin synthesis, fermentative synthesis of fructosylated chondroitin using the strain K4, followed by removal of the fructosyl groups via acidolysis and other methods to prepare chondroitin, has the characteristics of environmental friendliness, cost-effectiveness and the like.

In recent years, researchers at home and abroad have conducted systematic studies on the microbial fermentative production of fructosylated chondroitin from the perspectives of biochemical engineering and metabolic engineering. The patent with publication number CN104974973B has disclosed a technology for the fermentative production of chondroitin using a recombinant strain of E. coli. Specifically, genes kfoA and kfoC derived from E. coli K4 are introduced via a vector into an E. coli strain, such as K5, capable of producing UDP-glucuronic acid, where the gene kfoA encodes UDP-glucose-4-epimerase, the gene kfoC encodes chondroitin synthase, the vector contains an antibiotic resistance gene, and the chondroitin yield reaches 52.6 mg/L. The patent with publication number CN102869782B has disclosed a technology for the fermentative production of chondroitin using recombinant E. coli K12, E. coli B, or Xanthomonas campestris. Specifically, one or more genes selected from kfoA, kfoC, kfoF, kfoG, kfoB, kpsF, kpsE, kpsD, kpsU, kpsC, kpsS, kpsM or kpsT are introduced via a vector into an E. coli or Xanthomonas strain without one or more functional genes of kfoD, orf3(kfol), kfoE or orfl(kfoH). The recombinant strain is cultured in a medium with glycerol as a carbon source for 24-72 h, and 15-50 g/L of non-fructosylated chondroitin is secreted from cells. The patent with publication number CN103228781B has disclosed the use of genetic engineering measures to inactivate a glycosyltransferase gene kfoE, which adds fructosyl residues to a chondroitin main chain, to obtain recombinant derivatives (ΔkfoE/kan^{R}, DSM23578 and ΔkfoE, DSM23644) of E. coli K4 capable of directly producing non-fructosylated chondroitin backbone. However, since plasmid carriage impairs cell growth, and the instability of plasmid replication may also cause instability in chondroitin yield, the chondroitin yield of this strain has not been reported. The patent with publication number CN115895987B has enhanced the synthesis of a precursor UDP-GalNAc by increasing the copy numbers of genes g/mS and glmM in an E. coli genome, in which g/mS encodes aminotransferase and glmM encodes phosphoglucosamine mutase, which solves the problem of plasmid instability and increases the yield of fructosylated chondroitin; and the yield of fructosylated chondroitin reaches 7.12 g/L when the recombinant strain is fermented in a 7.5 L fermenter. Nevertheless, whether it is fructosylated chondroitin or non-fructosylated chondroitin backbone, the yields thereof have failed to meet the requirements of industrial production, and cannot replace the direct extraction method, so that further increasing the yield of chondroitin or the analog fructosylated chondroitin thereof has become an urgent problem to be solved.

### SUMMARY

In view of the problems in the prior art, such as plasmid instability in plasmid-containing recombinant strains and low yield of chondroitin or analogs thereof, the present disclosure provides a plasmid-free strain with high-yield production of chondroitin backbone or analogs thereof.

The present disclosure provides an engineered E. coli strain, which expresses one or more of the following mutants: a phosphotransferase mutant, a phosphoglucosamine mutase mutant, a glutamate synthase mutant, an NADH oxidoreductase mutant, and a lipid transport system substrate-binding protein mutant.

In one embodiment, the engineered E. coli strain expresses the phosphotransferase mutant, the phosphoglucosamine mutase mutant, the glutamate synthase mutant, the NADH oxidoreductase mutant, and the lipid transport system substrate-binding protein mutant.

In one embodiment, the engineered E. coli strain takes E. coli K4 (ATCC 23502) as an original strain, and expresses one or more of the following mutants: the phosphotransferase mutant, the phosphoglucosamine mutase mutant, the glutamate synthase mutant, the NADH oxidoreductase mutant, and the lipid transport system substrate-binding protein mutant.

In one embodiment, the phosphotransferase mutant includes an amino acid sequence as set forth in SEQ ID NO: 1; the phosphoglucosamine mutase mutant includes an amino acid sequence as set forth in SEQ ID NO: 3; the glutamate synthase mutant includes amino acid sequences as set forth in SEQ ID NO: 5 and SEQ ID NO: 7; the NADH oxidoreductase mutant includes an amino acid sequence as set forth in SEQ ID NO: 9; and the lipid transport system substrate-binding protein mutant includes an amino acid sequence as set forth in SEQ ID NO: 11.

In one embodiment, the engineered E. coli strain is E. coli DH001, which has been preserved on Sep. 26, 2024 at the China General Microbiological Culture Collection Center (CGMCC), with a preservation number of CGMCC No. 32081, the preservation address is Beijing, China, and the strain is classified and designated as E. coli.

In one embodiment, the E. coli DH001 expresses proteins indicated by one or more amino acid sequences as set forth in SEQ ID NO: 1, SEQ ID NO: 3, SEQ ID NO: 5, SEQ ID NO: 7, SEQ ID NO: 9 and SEQ ID NO: 11.

In one embodiment, the E. coli DH001 has genes with nucleotide sequences as set forth in SEQ ID NO: 2, SEQ ID NO: 4, SEQ ID NO: 6, SEQ ID NO: 8, SEQ ID NO: 10 and SEQ ID NO: 12.

The present disclosure also provides a composition containing the E. coli DH001.

In one embodiment, the composition includes, but is not limited to, a direct-vat-set (DVS) starter containing viable cells of the E. coli DH001.

The present disclosure also provides a method for improving fructosylated chondroitin production capacity of E. coli, which includes (a) or (b):
(a) expressing one or more of the following mutants in E. coli: a phosphoenolpyruvate (PEP) phosphotransferase mutant, a phosphoglucosamine mutase mutant, a glutamate synthase large subunit mutant, a glutamate synthase small subunit mutant, an NADH oxidoreductase mutant, and a lipid transport system substrate-binding protein mutant;
(b) mutating encoding sequences of one or more of the following proteins in E. coli: phosphoenolpyruvate (PEP)-dependent phosphotransferase glvB, phosphoglucosamine mutase glmM, glutamate synthase gltB and gltD, NADH oxidoreductase hcr, and lipid transport system substrate-binding protein mlaC.

In one embodiment, the method involves in-situ substitution of genes glvB, glmM, gltB, gltD, hcr, and mlaC in an E. coli genome.

In one embodiment, the E. coli includes, but is not limited to, E. coli K4 (ATCC 23502).

In one embodiment, the mutation involves mutating arginine (R) at position 224 of PEP-dependent phosphotransferase (glvB) to cysteine (C), and the amino acid sequence of the obtained mutant is as set forth in SEQ ID NO: 1.

In one embodiment, the mutation involves mutating alanine (A) at position 128 of phosphoglucosamine mutase (glmM) to threonine (T), and the amino acid sequence of the obtained mutant is as set forth in SEQ ID NO: 3.

In one embodiment, the mutation involves mutating proline (P) at position 223 of a large subunit gltB of glutamate synthase to serine (S), and the amino acid sequence of the obtained mutant is as set forth in SEQ ID NO: 5.

In one embodiment, the mutation involves mutating proline (P) at position 190 of a small subunit gltD of glutamate synthase to serine (S), and mutating glutamic acid (E) at position 286 to lysine (K), and the amino acid sequence of the obtained mutant is as set forth in SEQ ID NO: 7.

In one embodiment, the mutation involves mutating threonine (T) at position 174 of NADH oxidoreductase her to isoleucine (I), and the amino acid sequence of the obtained mutant is as set forth in SEQ ID NO: 9.

In one embodiment, the mutation involves mutating alanine (A) at position 88 of phospholipid transport system substrate-binding protein mlaC to valine (V), and mutating proline (P) at position 140 to serine (S), and the amino acid sequence of the obtained mutant is as set forth in SEQ ID NO: 11.

In one embodiment, the nucleotide sequence of the gene glvB encoding the phosphotransferase mutant is as set forth in SEQ ID NO: 2; the nucleotide sequence of the gene glmM encoding the phosphoglucosamine mutase mutant is as set forth in SEQ ID NO: 4; the nucleotide sequence of the gene gltB encoding the glutamate synthase large subunit mutant is as set forth in SEQ ID NO: 6; the nucleotide sequence of the gene gltD encoding the glutamate synthase small subunit mutant is as set forth in SEQ ID NO: 8; the nucleotide sequence of the gene her encoding the NADH oxidoreductase mutant is as set forth in SEQ ID NO: 10; and the nucleotide sequence of the gene mlaC encoding the phospholipid transport system substrate-binding protein mutant is as set forth in SEQ ID NO: 12.

In one embodiment, the original strain of the recombinant E. coli includes, but is not limited to, E. coli K4.

The present disclosure also provides a method for fermentative production of fructosylated chondroitin using the E. coli DH001.

In one embodiment, the method involves fermenting the E. coli in a medium for 40 h-48 h and collecting fructosylated chondroitin from a fermentation product.

In one embodiment, a carbon source in the medium includes, but is not limited to, glucose.

In one embodiment, a nitrogen source in the medium is an organic nitrogen source and/or an inorganic nitrogen source. Optionally, the organic nitrogen source may be yeast extract powder, and the inorganic nitrogen source may be ammonium sulfate.

In one embodiment, the nitrogen source is yeast extract powder and ammonium sulfate.

In one embodiment, the medium further contains inorganic salts and trace elements essential for strain growth, including but not limited to phosphorus, magnesium, iron, and the like.

In one embodiment, the inorganic salts and trace elements are selected from one or more of potassium dihydrogen phosphate, magnesium sulfate heptahydrate, and ferrous sulfate heptahydrate.

In one embodiment, the medium contains glucose, yeast extract powder, ammonium sulfate, potassium dihydrogen phosphate, and magnesium sulfate heptahydrate.

In one embodiment, the fermentation is carried out at 30-37° C.

In one embodiment, glucose is supplemented during the fermentation process.

In one embodiment, the method may further include a step of recovering the fructosylated chondroitin from a fermentation broth.

In one embodiment, the recovering is followed by purification.

In one embodiment, the recovering may be performed using centrifugation, filtration, ultrafiltration, ethanol precipitation, and various forms of chromatography such as gel filtration chromatography (molecular sieve), adsorption chromatography, ion exchange chromatography, or a combination thereof for recovery and purification. The recovering step and the purification step may be performed continuously or discontinuously in partial sequence, or may be performed simultaneously or by combining into one step, and the manner of the steps is not limited thereto.

The present disclosure also provides use of the E. coli DH001, or the composition, or the method in preparation of fructosylated chondroitin or related products thereof.

In one embodiment, the related products include, but are not limited to, chondroitin and chondroitin sulfate.

### Beneficial Effects

(1) On the basis of E. coli K4 (ATCC 23502), the present disclosure constructs the strain DH001 with enhanced fructosylated chondroitin synthesis capacity by mutating the key enzyme genes (PEP-dependent phosphotransferase glvB, phosphoglucosamine mutase glmM, glutamate synthase gltB and gltD, NADH oxidoreductase hcr, and lipid transport system substrate-binding protein mlaC) in a fructosylated chondroitin synthesis pathway.
(2) The strain constructed by the present disclosure exhibits excellent production performance and genetic stability. After 8 passages, the yield of fructosylated chondroitin in a 5 L fermenter remains at 8 g/L or above.
(3) The present disclosure provides the method for fermentative production of fructosylated chondroitin using the strain DH001. The yield of fructosylated chondroitin can reach 10 g/L in a 5 L fermenter. When the fermentation scale is expanded to a 100 L fermenter for high-density fermentation, the production cycle is properly controllable, and the yield of fructosylated chondroitin reaches 15-20 g/L. The obtained final product has a uniform structure and stable quality, which effectively reduces the production costs and the risk of environmental pollution, thus being suitable for large-scale industrial production applications.

### BRIEF DESCRIPTION OF FIGURES

FIG. 1 shows a synthesis pathway of fructosylated chondroitin (K4CPS).

FIG. 2 shows chondroitin yields of recombinant strains in shake-flask fermentation of Example 2.

FIG. 3A shows the sequencing result of a mutant gene glvB^{R224C} of a recombinant strain FCH5 in Example 2.

FIG. 3B shows the sequencing result of a mutant gene glmM^{A128T} of the recombinant strain FCH5 in Example 2.

FIG. 3C shows the sequencing result of a mutant gene gltB^{P223S} of the recombinant strain FCH5 in Example 2.

FIG. 3D shows the sequencing result of a mutant gene hcr^{T1741} of the recombinant strain FCH5 in Example 2.

FIG. 3E shows the sequencing result of a mutant gene gltD^{P190S}/E^{2S6K} of the recombinant strain FCH5 in Example 2.

FIG. 3F shows the sequencing result of a mutant gene mlaC^{A88V}/P^{140S} of the recombinant strain FCH5 in Example 2.

FIG. 4 is an HPLC analytical chromatogram of chondroitin disaccharide obtained after enzymolysis of a sodium chondroitin sulfate standard product.

FIG. 5 is an HPLC analytical chromatogram of chondroitin disaccharide obtained after acidolysis and enzymolysis of a fermentation product in Example 4.

FIG. 6A is an analytical chromatogram of a monosaccharide standard product.

FIG. 6B is an analytical chromatogram showing monosaccharide compositions of a fructosylated chondroitin sample prepared in Example 4.

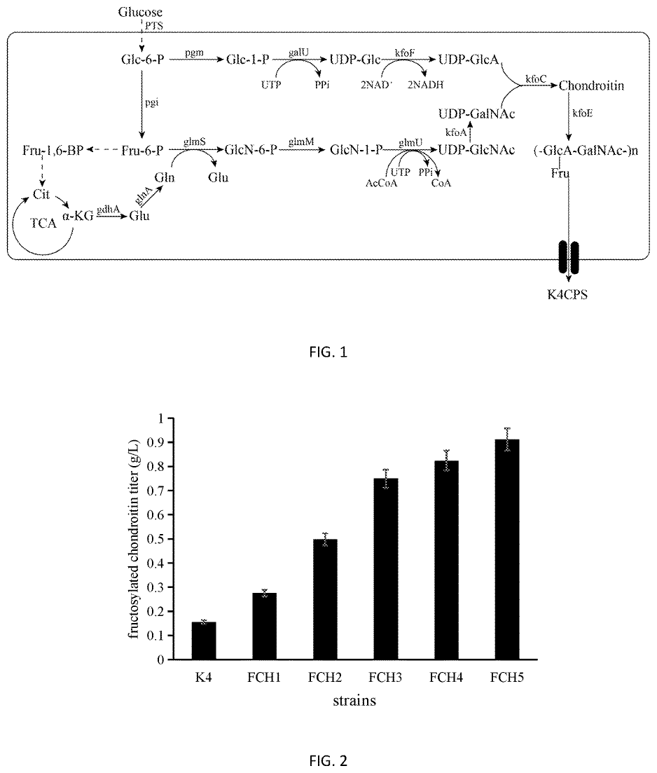

### DETAILED DESCRIPTION

(I) Sequence information:
(1) SEQ ID NO: 1 is the amino acid sequence of a mutant glvB;
(2) SEQ ID NO: 2 is the nucleotide sequence of a mutant gene glvB;
(3) SEQ ID NO: 3 is the amino acid sequence of a mutant glmM;
(4) SEQ ID NO: 4 is the nucleotide sequence of a mutant gene glmM;
(5) SEQ ID NO: 5 is the amino acid sequence of a mutant gltB;
(6) SEQ ID NO: 6 is the nucleotide sequence of a mutant gene gltB;
(7) SEQ ID NO: 7 is the amino acid sequence of a mutant gltD;
(8) SEQ ID NO: 8 is the nucleotide sequence of a mutant gene gltD;
(9) SEQ ID NO: 9 is the amino acid sequence of a mutant hcr;
(10) SEQ ID NO: 10 is the nucleotide sequence of a mutant gene hcr;
(11) SEQ ID NO: 11 is the amino acid sequence of a mutant mlaC;
(12) SEQ ID NO: 12 is the nucleotide sequence of a mutant gene mlaC;
(13) SEQ ID NO: 13 is the sgRNA sequence of E. coli with the gene glvB knocked out;
(14) SEQ ID NO: 14 is the sgRNA sequence integrating the mutant gene glvB;
(15) SEQ ID NO: 15 is the sgRNA sequence of E. coli with the gene glmM knocked out;
(16) SEQ ID NO: 16 is the sgRNA sequence integrating the mutant gene glmM;
(17) SEQ ID NO: 17 is the sgRNA sequence of E. coli with the gene gltB knocked out;
(18) SEQ ID NO: 18 is the sgRNA sequence integrating the mutant gene gltB;
(19) SEQ ID NO: 19 is the sgRNA sequence of E. coli with the gene gltD knocked out;
(20) SEQ ID NO: 20 is the sgRNA sequence integrating the mutant gene gltD;
(21) SEQ ID NO: 21 is the sgRNA sequence of E. coli with the gene hcr knocked out;
(22) SEQ ID NO: 22 is the sgRNA sequence integrating the mutant gene hcr;
(23) SEQ ID NO: 23 is the sgRNA sequence of E. coli with the gene mlaC knocked out; and
(24) SEQ ID NO: 24 is the sgRNA sequence integrating the mutant gene mlaC.

Unless otherwise specified, amino acid mutants are named using the accepted IUPAC single-letter amino acid abbreviations. For example, R224C denotes a mutant obtained by mutating arginine (R) at position 224 to cysteine (C) on the basis of the wild type.

### (II) Determination Method:

Fructosylated chondroitin yield detection: According to the analytical method described on Page 1595 of Volume II of Chinese Pharmacopoeia (2020 Edition): 10 mL of a fermentation broth was taken and added into a 50 mL centrifuge tube, pure glacial acetic acid was added to adjust the pH value to 3.8, the resulting mixture was placed in a water bath at 85° C. for acidolysis for 4 h and was cooled to the room temperature after acidolysis, 6 M sodium hydroxide (NaOH) was added to adjust the pH value to 7.0-8.0, and then the mixture was transferred to a 25 mL volumetric flask and brought to volume. 100 μL of a sample obtained after acidolysis was mixed with 100 μL of chondroitin sulfate ABC enzyme (SIGMA-Aldrich, C2905-10UN), 800 μL of a trihydroxymethylchloromethane buffer was added and fully mixed well, the resulting mixture was placed in a water bath at 37° C. for reaction for 1 h, heated in a boiling water bath for 5 min after enzymolysis, cooled in cold water and then centrifuged, and 20 μL of supernatant was taken and loaded into a HypersilSAX chromatographic column (Waters) for HPLC analysis, with a detection wavelength of 232 nm. Quantification was performed by an external standard method: The sum of peak areas of ΔDi-OS, ΔDi-4S, and ΔDi-6S after enzymolysis of a 10 g/L sodium chondroitin sulfate standard product (National Institutes for Food and Drug Control, 4386/10/12, Batch No. 140792-202003) was taken as a reference product peak area; the peak area of ΔDi-OS of the sample after enzymolysis was taken as a sample peak area to calculate the chondroitin content (sample peak area/reference product peak area×standard product concentration of 10 g/L×dilution factor of 2.5); and when calculating the fructosylated chondroitin content, the chondroitin content was multiplied by a coefficient of 1.43 (coefficient=molecular weight of fructosylated chondroitin disaccharide÷molecular weight of chondroitin disaccharide, i.e., 540÷378=1.43).

Glucose determination method: The fermentation broth was centrifuged at 10000 r/min for 5 min, then the supernatant was diluted 20 times, and a biosensor analyzer M-100 (Sieman Technology) was employed for determination.

Unless otherwise specified, the raw materials and reagents used in the following examples are commercially available products, or can be prepared by known methods, and the operations carried out are known in the art, or according to the user manuals of commercially available products.

### Example 1 Construction of Recombinant Strains

E. coli K4 (ATCC 23502) was subjected to gene editing using a Crispr-Cas9 system, and a codon encoding the amino acid at position 224 of the gene glvB was mutated from CGT to TGT, resulting in an E. coli strain FCH1; subsequently, a codon encoding the amino acid at position 128 of the gene glmM was mutated from GCC to ACC, resulting in an E. coli strain FCH2; then, a codon encoding the amino acid at position 223 of the gene gltB was mutated from CCG to TCG, a codon encoding the amino acid at position 190 of the gene gltD was mutated from CCG to TCG, and a codon encoding the amino acid at position 286 of the gene gltD was mutated from GAA to AAA, resulting in an E. coli strain FCH3; then, a codon encoding the amino acid at position 174 of the gene hcr was mutated from ACC to ATC, resulting in an E. coli strain FCH4; and then, a codon encoding the amino acid at position 88 of the gene mlaC was mutated from GCT to GTT, and a codon encoding the amino acid at position 140 of the gene mlaC was mutated from CCG to TCG, resulting in an E. coli strain FCH5. The specific steps are as follows:

(1) Preparation of E. coli K4/pCas Competent Cells

50 ng of pCas plasmid (purchased from Addgene) was transformed into E. coli K4 competent cells via heat shock at 42° C. for 90 s, and the transformed cells were spread on a solid LB medium (containing 100 mg/L kanamycin (kana)) and incubated overnight in an incubator at 30° C. to obtain an E. coli K4/pCas strain. Single colonies were picked and inoculated into 5 mL of a liquid LB medium (containing 100 mg/L kana), 50 μL of a 1 M L-arabinose (L-Ara) solution was added into the medium, and the mixture was incubated overnight at 30° C. and 180 rpm to obtain a seed fermentation broth. 500 μL of the seed fermentation broth was inoculated into 50 mL of the liquid LB medium (containing 100 mg/L kana), 500 μL of 1 M L-Ara was added into the medium, and the mixture was cultured at 30° C. and 180 rpm until OD₆₀₀ reached 0.4-0.6; the mixture was placed in an ice bath for 5 min, and the bacterial liquid was poured into a 50 mL sterile centrifuge tube on a super clean bench, and centrifuged at 8° C. and 5000 rpm for 10 min; the supernatant was discarded, and the cells were resuspended in 20 mL of pre-chilled 100 mM CaCl₂ and then placed in an ice bath for 30 min; after centrifugation at 8° C. and 5000 rpm for 10 min, the supernatant was discarded, and the cells were resuspended in 0.5 mL of pre-chilled 100 mM CaCl₂ and 60% glycerol, aliquoted into 1.5 mL sterile EP tubes at 50 μL per tube, and stored at −80° C.

(2) Knockout of Endogenous Gene glvB in E. coli

An upstream homologous arm fragment was obtained, and an amplification system was as follows: 1 μL of 50 ng/μL E. coli K4 genomic DNA serving as a template, 1 μL of each of 10 μM primers glvB-F1 and glvB-R1 (Table 1), 25 μL of 2×PrimeSTAR MAX Premix, and 2 μL of ddH₂O₂. PCR reaction conditions: Pre-denaturation at 98° C. for 5 min, followed by temperature cycling at 98° C. for 10 s, at 57° C. for 15 s, and at 72° C. for 1 min, with a total of 30 cycles; and the final temperature was 4° C. After PCR amplification was finished, the correct product was verified by 1% agarose gel electrophoresis, and the upstream homologous arm fragment was purified using a PCR Purification Kit.

A downstream homologous arm fragment was obtained using the same method as the upstream homologous arm fragment, with primers being glvB-F2 and glvB-R2 (Table 1). Overlap PCR was employed to obtain a Donor DNA, and an amplification system was as follows: 1 μL of each of 50 ng/μL upstream and downstream homologous arm fragments serving as templates, 1 μL of each of 10 μM primers glvB-F2 and glvB-R2, 25 μL of 2×PrimeSTAR MAX Premix, and 2 μL of ddH₂O₂. PCR reaction conditions: Pre-denaturation at 98° C. for 5 min, followed by temperature cycling at 98° C. for 10 s, at 57° C. for 15 s, and at 72° C. for 1 min, with a total of 30 cycles; and the final temperature was 4° C.

Based on the CRISPR/Cas9 gene editing principle, a specific guided SgRNA sequence as set forth in SEQ ID NO: 13 was employed, and pTarget-ΔglvB plasmids were obtained through synthesis by Genewiz Biotechnology Co., Ltd. (Suzhou) and ligation into pTarget plasmids.

Competent cells carrying pCas plasmids were taken at −80° C. and allowed to stand still in an ice bath for 2 min; and then, 500 ng of Donor DNA and 50 ng of pTarget-ΔglvB were added, mixed gently, and allowed to stand still on ice for 30 min. After that, the resulting mixture was subjected to heat shock at 42° C. for 90 s and then allowed to stand still in an ice bath for 2 min; and then, 1 mL of a pre-chilled LB medium was added to the competent cells, the cells were placed in a shaker at 30° C. for incubation at 180 rpm for 2 h, and 100 μL of the culture was spread on a solid LB medium (containing 50 mg/L streptomycin (strep) and 100 mg/L kana), and incubated overnight at 30° C.

Several monoclonal colonies were picked from a transformation plate to serve as templates for colony PCR verification of positive clones, with glvB-TF/glvB-TR used as primers, and the size of the amplified fragment was 1762 bp.

The positive clones were inoculated into 5 mL of a liquid LB medium (100 mg/L kana), and IPTG with the final concentration of 0.1 mM was added to induce pCas plasmids to eliminate pTarget-ΔglvB plasmids; and the culture was incubated at 30° C. and 180 rpm for 16 h, then streaked onto a solid LB medium (100 mg/L kana), and incubated overnight at 30° C. Single colonies were picked (only half of each single colony was taken), numbered, and inoculated onto the corresponding numbered regions of a solid LB medium (50 mg/L strep) according to the numbers, followed by overnight incubation at 30° C., and the single colonies unable to grow on the medium were identified as strains with successful elimination of the pTarget-ΔglvB plasmids.

(3) Integration of Mutant Gene glvB^{R224C}

The strains with pTarget-ΔglvB eliminated prepared in step (2) were re-prepared into competent cells carrying pCas plasmids.

The E. coli K4 genomic DNA was used as a template, and upstream and downstream homologous arms were amplified with primer pairs glvB-F3/glvB-R3 and glvB-F4/glvB-R4, respectively; a glvB^{R224C} gene fragment (SEQ ID NO: 2) was synthesized by Genewiz Biotechnology Co., Ltd. (Suzhou); and by using the upstream and downstream homologous arms and a glvB^{R224C} gene sequence as templates and glvB-F3/glvB-R4 as a template, the three fragments were fused by Overlap PCR to generate Donor DNA. Referring to step (2) for the PCR system and method.

Based on the CRISPR/Cas9 gene editing principle, a specific guided SgRNA sequence as set forth in SEQ ID NO: 14 was employed, and pTarget-glvB^{R224C} plasmids were obtained through synthesis by Genewiz Biotechnology Co., Ltd. (Suzhou) and ligation into pTarget plasmids.

Referring to step (2) for other operations. Positive clone identification primers are glvB TF/glvB TR, and the size of the amplified fragment was 3073 bp.

Subsequent gene editing was performed following the aforementioned operation procedure for the implementation of editing of genes glmM, gltB, gltD, hcr, and mlaC. The relevant primers are listed in Table 1. The sizes of the amplified fragments identified by PCR for positive clones of knockout/mutant gene integration of the genes glmM, gltB, gltD, hcr, and mlaC were 1687/2721 bp, 1167/2427 bp, 1734/2808 bp, 1371/2340 bp, and 1180/1816 bp, respectively.

Upon completion of all gene editing, the pCas plasmids were eliminated to finalize strain construction.

TABLE 1
Primers and sequences

| Gene | Primer | Sequence (5′→3′) |
|---|---|---|
| glvB | glvB-F1 | AATGGCCGGAGGCGCAGGCAT (SEQ ID NO: 25) |
| | glvB-R1 | AACCACTGAGAATTTGGTCATTCCACATCCTCTTTTCTCAATTC (SEQ ID NO: 26) |
| | glvB-F2 | TGACCAAATTCTCAGTGGTTGTCG (SEQ ID NO: 27) |
| | glvB-R2 | GGCTTTCAATACCGATTGGCATG (SEQ ID NO: 28) |
| | glvB-TF | CGCGGATGTTAGCCAAAATGAAT (SEQ ID NO: 29) |
| | glvB-TR | CTTCAATTGATGTCCACCAGCC (SEQ ID NO: 30) |
| | glvB-F3 | CACCTGGTATTCGCTGGCAT (SEQ ID NO: 31) |
| | glvB-R3 | CGTTGAATTTGACTGAGCATAAGCATTGCGGAGCGGTTAAGAATTC (SEQ ID NO: 32) |
| | glvB-F4 | CCATTACGGAGGCAGTATAATGACCAAATTCTCAGTGGTTGTCG (SEQ ID NO: 33) |
| | glvB-R4 | GGCTTTCAATACCGATTGGCATG (SEQ ID NO: 34) |
| glmM | glmM-F1 | GGATATCGCTGGTCGGCTTAGC (SEQ ID NO: 35) |
| | glmM-R1 | TGCAAAGGAAAACAAACGCTAGCGTTAGATAACTGGCTAAAAAGG (SEQ ID NO: 36) |
| | glmM-F2 | AGCGTTTGTTTTCCTTTGCAGAC (SEQ ID NO: 37) |
| | glmM-R2 | ACATCCAAACCAGAAGTCATCCG (SEQ ID NO: 38) |
| | glmM-TF | ATCCATATCAGACCGAAACACTG (SEQ ID NO: 39) |
| | glmM-TR | GGCGGCGGAAGTTAGCGTTG (SEQ ID NO: 40) |
| | glmM-F3 | CCGGAACTGGAGTTGACACAA (SEQ ID NO: 41) |
| | glmM-R3 | ATGCAGTAAAAGCCGTTTAAAGGCGCTGATATGGGAGCC (SEQ ID NO: 42) |
| | glmM-F4 | AAATATTTACGATTACTCATAGCGTTTGTTTTCCTTTGCAGAC (SEQ ID NO: 43) |
| | glmM-R4 | ACATCCAAACCAGAAGTCATCCG (SEQ ID NO: 44) |
| gltB | gltB-F1 | GTGCGCGGGATGATTCCAGT (SEQ ID NO: 45) |
| | gltB-R1 | GTTAACTGAGAACAAACTAATCGTCTGAAAGCTGGCAGCC (SEQ ID NO: 46) |
| | gltB-F2 | TTAGTTTGTTCTCAGTTAACAATTCATAT (SEQ ID NO: 47) |
| | gltB-R2 | ATTTTTTCAGGCCGGATATGGC (SEQ ID NO: 48) |
| | gltB-TF | CCGCGCGTCTGTTTGAGGC (SEQ ID NO: 49) |
| | gltB-TR | ATTTTTTCAGGCCGGATATGGC (SEQ ID NO: 50) |
| | gltB-F3 | GGTAAAAACGTCGGCGACGATC (SEQ ID NO: 51) |
| | gltB-R3 | AGCGTGTGAAAGGCGAGTAATACTTTCCATCTTTCCTCACTCCTTAAGTTAG (SEQ ID NO: 52) |
| | gltB-F4 | TGAACACGAAATTTATCCATTTAGTTTGTTCTCAGTTAACAATTCATAT (SEQ ID NO: 53) |
| | gltB-R4 | ATTTTTTCAGGCCGGATATGGC (SEQ ID NO: 54) |
| gltD | gltD-F1 | TTTCGGCGTGCGTAACTCCG (SEQ ID NO: 55) |
| | gltD-R1 | AGGCGGCGCTGGGACAGAGCTGTTGCTACCCCTTACTGCGC (SEQ ID NO: 56) |
| | gltD-F2 | GCTCTGTCCCAGCGCCGCCT (SEQ ID NO: 57) |
| | gltD-R2 | AATTCGCCAGACACTGTCTGG (SEQ ID NO: 58) |
| | gltD-TF | AGACTGCCGAACCGCATCCA (SEQ ID NO: 59) |
| | gltD-TR | TTCCACCTCTAGAAGGTCGTG (SEQ ID NO: 60) |
| | gltD-F3 | TGATAACGGCCTGCTGAACG (SEQ ID NO: 61) |
| | gltD-R3 | TGATAAACATTCTGACTCATGCAGACGATACCACCCGTC (SEQ ID NO: 62) |
| | gltD-F4 | TGAACTGGCTGGAAGTITAAGCTCTGTCCCAGCGCCGCCT (SEQ ID NO: 63) |
| | gltD-R4 | AATTCGCCAGACACTGTCTGG (SEQ ID NO: 64) |
| hcr | hcr-F1 | GGTGAGCCGTGAAAAACTGCGT (SEQ ID NO: 65) |
| | hcr-R1 | TATCATCGGGCATAAGGCGACGACAAACTCCTTACGCGCT (SEQ ID NO: 66) |
| | hcr-F2 | TCGCCTTATGCCCGATGATATTCCT (SEQ ID NO: 67) |
| | hcr-R2 | GCAATCGCCAGTACTTGTGGGA (SEQ ID NO: 68) |
| | hcr-TF | TGGATGCAGGCGAAACCGGT (SEQ ID NO: 69) |
| | hcr-TR | GCATGATACCAGTGCATGGT (SEQ ID NO: 70) |
| | hcr-F3 | GCGAAATGCTGCCTGCGCATG (SEQ ID NO: 71) |
| | hcr-R3 | TTGATTCGTTGGCATCGTCATAGACGCGGCAGACCTTCGA (SEQ ID NO: 72) |
| | hcr-F4 | GGGATTTGGTTCTCGCATAATCGCCTTATGCCCGATGATATTCCT (SEQ ID NO: 73) |
| | hcr-R4 | GCAATCGCCAGTACTTGTGGGA (SEQ ID NO: 74) |
| mlaC | mlaC-F1 | CTGAAAGTGGCTGCCATCGC (SEQ ID NO: 75) |
| | mlaC-R1 | AATTTCAGGAGAACCGACGCTGAGCGAGTCACTGAGCTGGA (SEQ ID NO: 76) |
| | mlaC-F2 | GCGTCGGTTCTCCTGAAATTAT (SEQ ID NO: 77) |
| | mlaC-R2 | TATTAGCAGCACTGCTGGCGG (SEQ ID NO: 78) |
| | mlaC-TF | TTAGTTTGTTCTCAGTTAACAATTCATAT (SEQ ID NO: 79) |
| | mlaC-TR | ATTTCGTTGTTTAACGGCTACG (SEQ ID NO: 80) |
| | mlaC-F3 | TTAGTTTGTTCTCAGTTAACAATTCATAT (SEQ ID NO: 81) |
| | mlaC-R3 | CTCTGGAAGAGAAAAAATAAATTTTTTCAGGCCGGATATGGC (SEQ ID NO: 82) |
| | mlaC-F4 | ATCATTAAACGTTTAAACATGCGTCGGTTCTCCTGAAATTAT (SEQ ID NO: 83) |
| | mlaC-R4 | TATTAGCAGCACTGCTGGCGG (SEQ ID NO: 84) |

### Example 2 Shake-Flask Fermentation of Recombinant Strains FCH1, FCH2, FCH3, FCH4, and FCH5

Shake-flask fermentation was performed on the E. coli strains FCH1, FCH2, FCH3, FCH4, and FCH5 constructed in Example 1. Monoclonal colonies of each recombinant strain were picked and inoculated into a 250 mL baffled shake flask containing 50 mL of a seed medium (with the formulation as shown in Table 2), followed by incubation at 37° C. and 200 rpm for 10 h. The seed fermentation broth was transferred at an inoculation amount (v/v) of 1% into a 500 mL baffled shake flask containing 100 mL of a fermentation medium (with the formulation as shown in Table 3). Fermentation conditions were: rotation speed: 200 rpm, temperature: 37° C., initial pH controlled at 7.5, and fermentation duration: 48 h. Each strain was tested in triplicate. Upon completion of fermentation, fermentation broths were collected, and fructosylated chondroitin yields were determined by HPLC. The E. coli K4 (ATCC 23502) was used as a control strain.

TABLE 2
Seed medium formulation

| Component | Content (g/L) |
|---|---|
| Glucose | 10 |
| Yeast extract powder | 2 |
| Ammonium sulfate | 1 |
| Potassium dihydrogen phosphate | 10 |
| Magnesium sulfate heptahydrate | 5 |
| pH | 7.5 |

TABLE 3
Fermentation medium formulation

| Component | Content (g/L) |
|---|---|
| Glucose | 15 |
| Yeast extract powder | 5 |
| Ammonium sulfate | 2 |
| Magnesium sulfate heptahydrate | 5 |
| Potassium dihydrogen phosphate | 10 |
| Ferrous sulfate heptahydrate | 0.3 |
| Sodium chloride | 10 |
| pH | 7.5 |

The fructosylated chondroitin yields of the strains in shake-flask fermentation are shown in FIG. 2. After 48 h of fermentation, the yields of the original strain K4 and the recombinant strains FCH1, FCH2, FCH3, FCH4, and FCH5 were 0.156 g/L, 0.275 g/L, 0.498 g/L, 0.749 g/L, 0.824 g/L, and 0.912 g/L, respectively. All the recombinant strains showed increased yields compared with the original strain, among which FCH5 exhibited a 484.6% increase relative to the original strain.

### Example 3 Genotype Analysis of Recombinant Strains

Genomic analysis was performed on the E. coli strain FCH5 constructed in Example 1. The specific steps are as follows:

### (1) Polymerase Chain Reaction-Single Strand Conformation Polymorphism Analysis (PCR-SSCP)

The genome of the strain FCH5 was extracted and subjected to PCR amplification using primer pairs glvB-TF/glvB-TR, glmM-TF/glmM-TR, gltB-TF/gltB-TR, gltD-TF/gltD-TR, hcr-TF/hcr-TR, and mlaC-TF/mlaC-TR, respectively. Referring to step (2) of Example 1 for the PCR system and method. After high-temperature denaturation and ice-bath incubation, the PCR amplification products were subjected to SSCP electrophoresis with 8% non-denatured polyacrylamide gel. The synthesized mutant gene was used as a positive control, a native wild-type gene was used as a negative control, and water was used as a blank control. Due to the presence of single or multiple base mutations in the mutant gene, the conformation of the mutant gene is different from that of the native gene, resulting in different migration rates in SSCP electrophoresis, and the final fragment band is inconsistent with the position of the negative control fragment, but consistent with the position of the positive control fragment, indicating that it is a strain with successful allelic substitution.

### (2) Sequencing

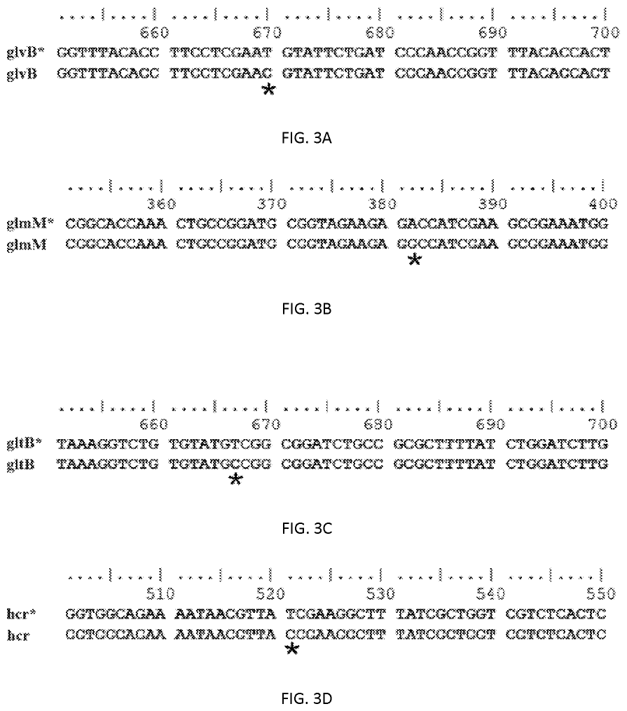

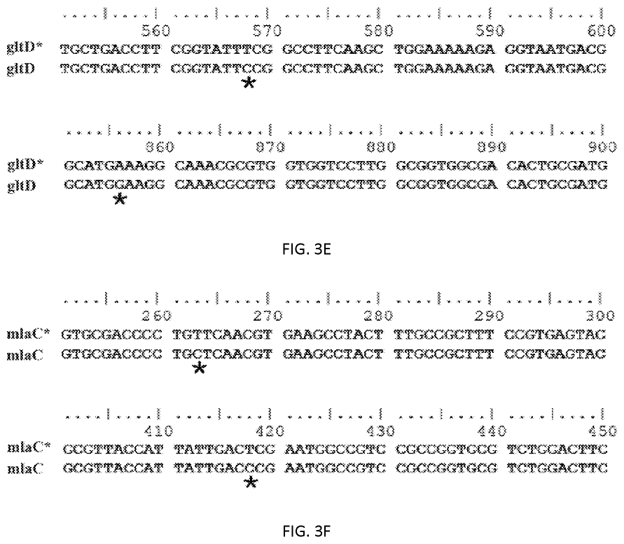

The PCR amplification products in step (1) were ligated into a pMD19-T vector for sequencing, and the successful allelic substitution of the strain was verified by sequence alignment of mutant bases. The results are shown in FIG. 3.

The strain FCH5 with correct genotype editing verified above was preserved and named DH001.

### Example 4 Fermentation of Recombinant Strain in 5 L Fermenter

Recombinant E. coli DH001 was fermented in a 5 L fermenter to produce fructosylated chondroitin, and E. coli K4 (ATCC 23502) was used as a control. The specific steps are as follows:

The E. coli K4 (ATCC 23502) and the recombinant strain DH001 were respectively inoculated into liquid LB mediums and activated overnight at 37° C., each of which was transferred at an inoculation amount (v/v) of 1% into a 500 mL baffled shake flask containing 100 mL of the seed medium as shown in Table 2, followed by incubation at 37° C. and 200 rpm for 10 h; the seed fermentation broth was transferred at an inoculation amount (v/v) of 10% into a 5 L T&J Intelli-FermA bench-top fermenter (purchased from T&J Bio-engineering (Shanghai) Co., Ltd.) containing the fermentation medium as shown in Table 3; and an antifoam agent was added at 0.2 g/L, and fermentation was carried out for 48 h according to the control process shown in Table 4. Each strain was tested in triplicate. Upon completion of fermentation, fermentation broths were collected, and fructosylated chondroitin yields were determined by HPLC. The results are shown in Table 5.

TABLE 4
Fermentation control process

| | |
|---|---|
| DO Calibration | Temperature: 37° C., aeration rate: 1 vvm, rotation speed: 1000 rpm, |
| (100%) | tank pressure: 0 MPa, calibration after 5 min |
| Culture | 37° C. |
| temperature | |
| pH | 7.5 ± 0.05 |
| Initial conditions: | Temperature: 37° C., pH: 6.8, tank pressure: 0.03 MPa, aeration rate: 1 |
| | vvm, rotation speed: 300 rpm |
| Whole-process | 1. When dissolved oxygen is less than 30%, increase stepwise: rotation |
| control | speed 500 rpm→700 rpm→800 rpm→ aeration rate 2 vvm→900 |
| | rpm→1000 rpm |
| | 2. Raise tank pressure to 0.05 MPa at 6 h of fermentation, and to 0.08 |
| | MPa at 12 h of fermentation |
| Residual sugar | Residual sugar should be controlled at 1-2 g/L in the initial 12 h of |
| control | fermentation, and at 0.5-1 g/L after 12 h of fermentation according to |
| | DO requirements |
| Fed-batch | 25% (w/v) ammonia water, 50% (w/v) glucose, 10% (v/v) antifoam agent |
| materials | |

TABLE 5
Fructosylated chondroitin fermentation results

| | Fructosylated chondroitin | |
|---|---|---|
| Strain | yield (g/L) | OD₆₀₀ |
| K4 | 1.91 ± 0.08 | 64.7 ± 1.6 |
| DH001 | 9.76 ± 0.24 | 87.3 ± 2.4 |

### Example 5 Stability Verification of Recombinant Strain DH001

The recombinant strain DH001 was subjected to fructosylated chondroitin fermentation according to Example 4. After fermentation, an appropriate amount of fermentation broth was collected, diluted with sterile water and then spread on a solid LB plate, and cultured at 37° C. until single colonies appeared. The single colonies were picked and verified by PCR for fructosylated chondroitin fermentation of the next passage. Continuous serial subculture and fermentation were conducted for 8 passages, and the fructosylated chondroitin yield in the fermentation broth of each passage was determined. The results showed that the fructosylated chondroitin yield remained at 8 g/L or above after 8 serial passages.

### Example 6 Fermentation of Recombinant Strain DH001 in 100 L Fermenter

### (1) Fermentation in Seed Tank

After the recombinant E. coli DH001 was activated in an LB medium according to the method of Example 3, it was inoculated into a 30 L seed tank at an inoculation amount of 5%. The seed medium formulation is shown in Table 6. The strain was cultured for 8-10 h until OD₆₀₀ reached 2.5-5.

TABLE 6
Seed tank medium formulation

| Component | Content (g/L) |
|---|---|
| Glucose | 5-10 |
| Yeast extract powder | 0.8-2 |
| Ammonium sulfate | 0.5-1 |
| Potassium dihydrogen phosphate | 10-12 |
| Magnesium sulfate heptahydrate | 3-5 |
| Antifoam agent | 0.2-0.3 |
| pH | 6.8-7.2 |

### (2) Fermentation in 100 L Fermenter

The seed fermentation broth prepared in step (1) was transferred at an inoculation amount of 10% into a 100 L fermenter. The fermentation medium formulation is shown in Table 7. The agitation speed was 300 rpm, and the aeration rate was 1 vvm, with dissolved oxygen calibration of 100%; the dissolved oxygen was maintained at 15%-30% throughout fermentation, the pH was controlled at 7.0-8.0 with 25% (w/v) ammonia water, the aeration rate was 1 vvm, and glucose with the mass fraction of 40%-60% was used as a fed-batch carbon source; and the glucose concentration in the fermentation system was enabled to be 1-2 g/L within the initial 12 h of fermentation, and the glucose concentration was controlled to 0.5-1 g/L after 12 h of fermentation in combination with DO requirements. Fermentation was carried out for 42-48 h according to the control process as shown in Table 4 until OD₆₀₀ reached 110-135, the fermenter was opened, the fermentation broth was collected, and the fructosylated chondroitin yield was determined.

TABLE 7
Fermentation medium formulation

| Component | Content (g/L) |
|---|---|
| Glucose | 10-15 |
| Yeast extract powder | 2-5 |
| Ammonium sulfate | 1.5-2.5 |
| Magnesium sulfate heptahydrate | 4-8 |
| Potassium dihydrogen phosphate | 5-10 |
| Ferrous sulfate heptahydrate | 0.1-0.3 |
| Sodium chloride | 5-10 |
| Antifoam agent | 0.2-0.5 |
| pH | 6.8-7.2 |

### (3) Fructosylated Chondroitin Yield Determination

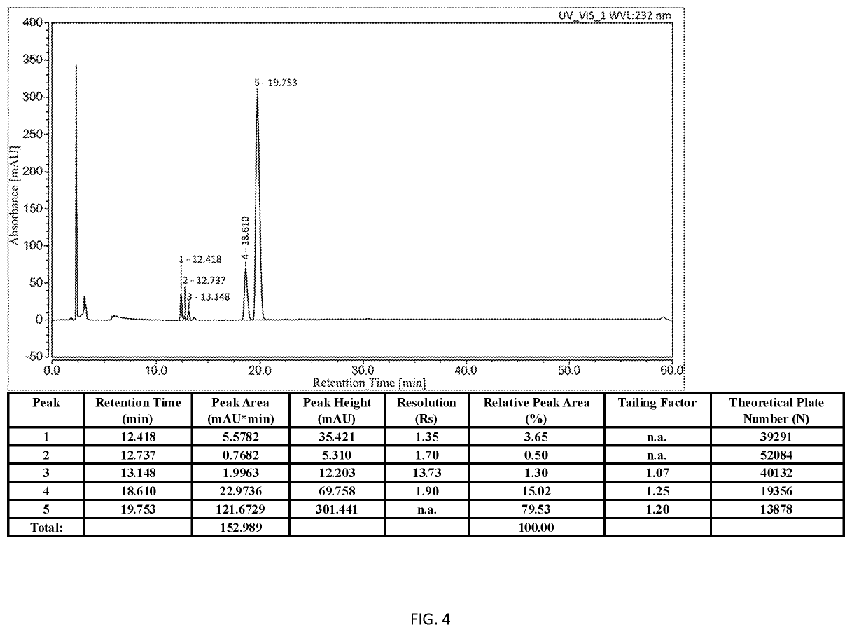

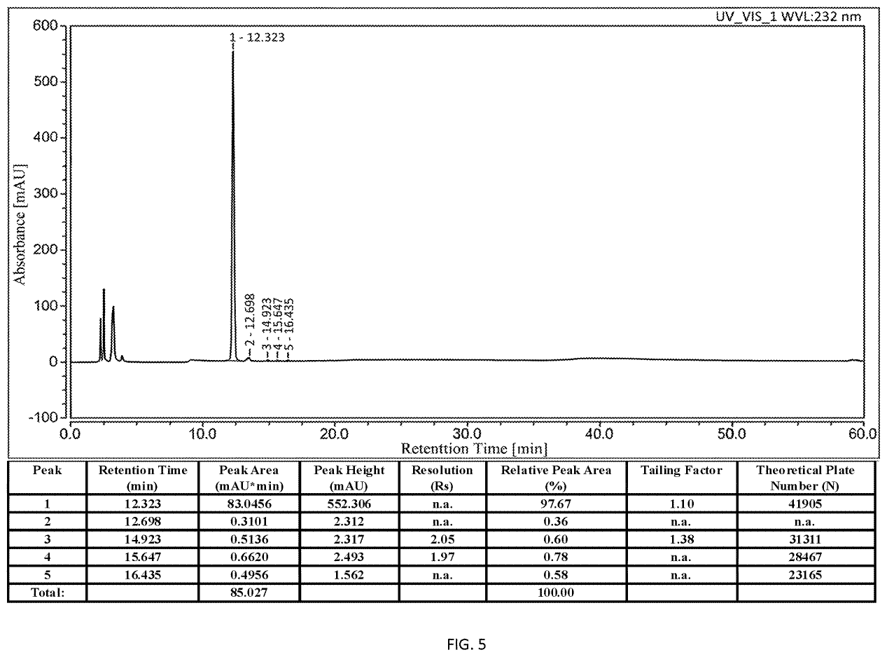

The fructosylated chondroitin content in the fermentation broth was determined by HPLC. The chromatogram of chondroitin disaccharide obtained after enzymolysis of a sodium chondroitin sulfate standard product is shown in FIG. 4, and the chromatogram of chondroitin disaccharide obtained after acidolysis and enzymolysis of the fermentation product fructosylated chondroitin is shown in FIG. 5. After calculation, the content of chondroitin disaccharide in the fermentation broth was 12.15±1.67 g/L, and the yield of fructosylated chondroitin was 17.37±2.39 g/L.

### (4) Analysis on Monosaccharide Compositions of Fructosylated Chondroitin

The monosaccharide compositions of fructosylated chondroitin recovered and purified from the fermentation broth were determined by HPLC based on 1-phenyl-3-methyl-5-pyrazolone (PMP) pre-column derivatization.

Hydrolysis: 2 mg of fructosylated chondroitin was dissolved in 2 mL of 2 M trifluoroacetic acid (TFA), hydrolyzed in a water bath at 100° C. for 2 h, adjusted to pH 7.0 with 6 M NaOH, and transferred to a 10 mL volumetric flask and brought to volume.

Derivatization: 200 μL of a sample was taken, added with 100 μL of 0.3 M NaOH and 100 μL of 0.5 M PMP-methanol, vortexed for uniform mixing, and placed in a water bath at 70° C. for hydrolysis for 30 min; and the product was cooled to the room temperature, added with 100 μL of 0.3 M hydrochloric acid (HCl) to neutralize NaOH, and then vortexed for uniform mixing.

Extraction: 1 mL of chloroform was added for extraction, centrifugation was performed at 5000 rpm for 3 min, and the supernatant was collected; and the extraction was repeated for three times to remove the excess PMP reagent as completely as possible.

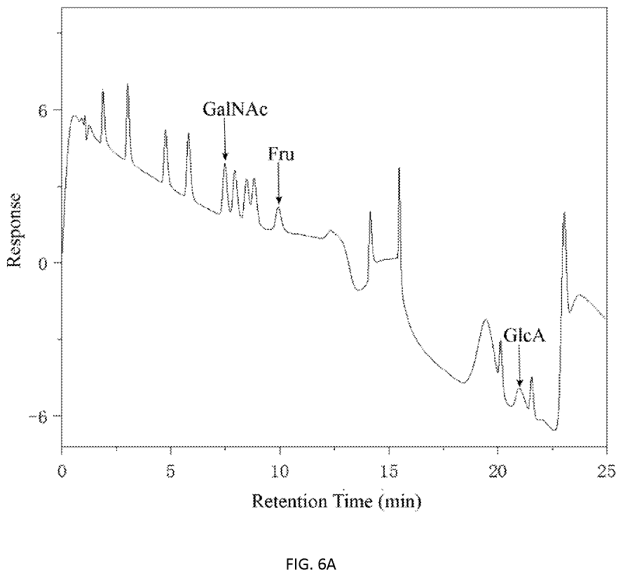

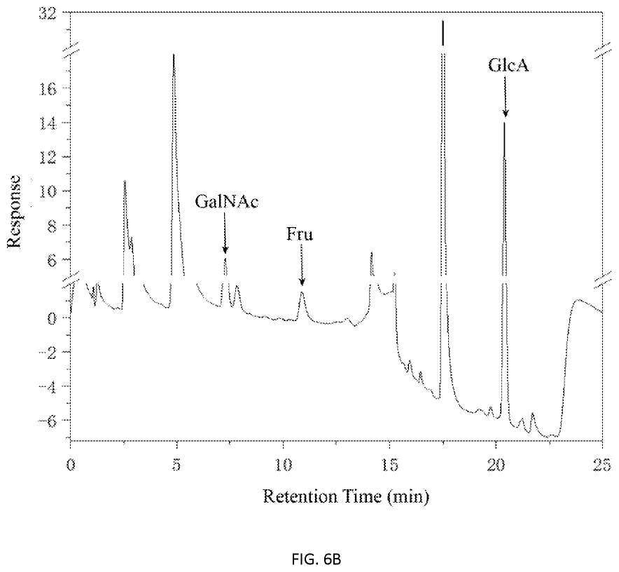

Detection: 10 μL of a sample was loaded into an HPLC, with a mobile phase of 0.1 M ammonium acetate-acetonitrile (at a volume ratio of 83:17), the column temperature of 35° C., and a detection wavelength of 254 nm, and the monosaccharide compositions were determined by using the retention time of a reference standard product. The results are shown in FIG. 6. The fructosylated chondroitin obtained through fermentation was composed of monosaccharides such as fructose, glucuronic acid, and N-acetylgalactose.

Although the present disclosure has been disclosed as above in exemplary examples, it is not limited by the foregoing examples. Those skilled in the art can make various changes and modifications without departing from the spirit and scope of the present disclosure. The protection scope of the present disclosure shall be as defined by the claims.

What is claimed is:

1. An engineered Escherichia coli strain, expressing one or more of the following mutants: a phosphotransferase mutant, a phosphoglucosamine mutase mutant, a glutamate synthase mutant, an NADH oxidoreductase mutant, and a lipid transport system substrate-binding protein mutant.

2. The engineered E. coli strain according to claim 1, wherein the engineered E. coli strain takes E. coli K4 as an original strain.

3. The engineered E. coli strain according to claim 1 wherein the phosphotransferase mutant comprises the amino acid sequence as set forth in SEQ ID NO:1; the phosphoglucosamine mutase mutant comprises the amino acid sequence as set forth in SEQ ID NO:3; the glutamate synthase mutant comprises the amino acid sequences as set forth in SEQ ID NO:5 and SEQ ID NO:7; the NADH oxidoreductase mutant comprises the amino acid sequence as set forth in SEQ ID NO:9; and the lipid transport system substrate-binding protein mutant comprises the amino acid sequence as set forth in SEQ ID NO:11.

4. The engineered E. coli strain according to claim 1, wherein the strain is E. coli DH001, preserved on Sep. 26, 2024 at the China General Microbiological Culture Collection Center (CGMCC), with a preservation number of CGMCC No. 32081, the preservation address is Beijing, China, and the strain is classified and designated as E. coli.

5. A method for improving fructosylated chondroitin production capacity of E. coli, comprising (a) or (b):
(a) expressing one or more of the following mutants in E. coli: a phosphotransferase mutant, a phosphoglucosamine mutase mutant, a glutamate synthase mutant, an NADH oxidoreductase mutant, and a lipid transport system substrate-binding protein mutant;
(b) mutating encoding sequences of one or more of the following proteins in E. coli: phosphotransferase, phosphoglucosamine mutase, glutamate synthase, NADH oxidoreductase, and lipid transport system substrate-binding protein.

6. The method according to claim 5, wherein the mutation is selected from one or more of the following:
(1) mutating arginine at position 224 of phosphotransferase to cysteine;
(2) mutating alanine at position 128 of phosphoglucosamine mutase to threonine;
(3) mutating proline at position 223 of a large subunit of glutamate synthase to serine;
(4) mutating proline at position 190 of a small subunit of glutamate synthase to serine, and mutating glutamic acid at position 286 to lysine;
(5) mutating threonine at position 174 of NADH oxidoreductase to isoleucine; and
(6) mutating alanine at position 88 of phospholipid transport system substrate-binding protein to valine, and mutating proline at position 140 to serine.

7. The method according to claim 5, wherein the E. coli comprises E. coli K4.

8. A method for preparing fructosylated chondroitin by fermentation, wherein the engineered E. coli strain according to claim 1 is fermented in a medium for 40 hours-48 hours, and fructosylated chondroitin is collected from a fermentation product.

9. The method according to claim 8, wherein a carbon source in the medium comprises glucose; and a nitrogen source comprises yeast extract powder and ammonium sulfate.

10. The method according to claim 9, wherein the fermentation is carried out at 30-37° C.

11. The method according to claim 10, wherein glucose is further supplemented during the fermentation process.

12. The method according to claim 11, further comprising a step of recovering the fructosylated chondroitin from a fermentation broth.

13. A method for preparing fructosylated chondroitin by fermentation, wherein the engineered E. coli strain according to claim 4 is fermented in a medium for 40 hours-48 hours, and fructosylated chondroitin is collected from a fermentation product.
